(19) United States
(12) Patent Application Publication
(10) Pub. No.: US 2023/0173140 A1
(43) Pub. Date: Jun. 8, 2023

(54) DOUBLE-LAYER OSTEOCHONDRAL TISSUE REPAIR STENT AND PREPARATION METHOD THEREFOR

(71) Applicant: SHAANXI GIANT BIOTECHNOLOGY CO., LTD, Shaanxi (CN)

(72) Inventors: Daidi FAN, Shaanxi (CN); Kaiqiang LIU, Shaanxi (CN); Zhiguang DUAN, Shaanxi (CN); Jianya YAN, Shaanxi (CN)

(73) Assignee: SHAANXI GIANT BIOTECHNOLOGY CO., LTD, Shaanxi (CN)

(21) Appl. No.: 17/922,125

(22) PCT Filed: Apr. 29, 2021
(86) PCT No.: PCT/CN2021/091001

§ 371 Date: Oct. 28, 2022

(30) Foreign Application Priority Data
Apr. 29, 2020 (CN) 202010357320.8

(51) Int. Cl. A61L 27/48

(52) U.S. Cl. CPC A61L 27/48; A61L 2430/06; A61L 2430/02; B29C 39/38

(57) ABSTRACT

Provided in the present application is a preparation method for a double-layer osteochondral tissue repair stent, comprising: formulating a first feed solution, the first feed solution comprising recombinant collagen, sodium hyaluronate, and hydroxyapatite; formulating a second feed solution, the second feed solution comprising recombinant collagen and sodium hyaluronate; freeze-drying the first feed solution and the second feed solution and forming a gel-like double-layer structure; and adding the gel-like double-layer structure into a crosslinking agent for crosslinking. The present method also relates to a double-layer osteochondral tissue repair stent, comprising: a first layer composed of raw materials including recombinant collagen, sodium hyaluronate, and hydroxyapatite; and a second layer composed of raw materials including recombinant collagen and sodium hyaluronate. The double-layer osteochondral tissue repair stent prepared by the present application has excellent mechanical properties, good biocompatibility, and a suitable degradation rate and, after degradation, the stent material can be reused as raw material for the formation of new bone, thus implementing osteochondral tissue repair.

### FIELD OF THE INVENTION

The present application belongs to the technical field of biomedical materials, in particular to a double-layer osteochondral tissue repair stent and a preparation method thereof.

### BACKGROUND OF THE INVENTION

Osteochondral defects are common diseases of joints, including articular cartilage and subchondral bone defects. There are many causes of osteochondral injuries, such as sports injuries, traffic accidents and various degenerative diseases. In addition, with the growth of age, the natural wear of cartilage tissue is easy to cause osteoarthritis, which is also the main cause of osteochondral injury. Because of physiological characteristics, cells in cartilage tissue can hardly regenerate, so cartilage reconstruction is a huge challenge. With the development of cartilage injury, it usually extends to subchondral bone, making the repair of cartilage and subchondral tissue a necessary condition. Articular cartilage and subchondral bone are closely connected in physiological structure and interact with each other to form interdependent functional units. It is reported that cartilage cannot be spontaneously repaired without the support of healthy subchondral bone. Therefore, for osteochondral diseases, subchondral bone should be repaired at the same time to reconstruct the cartilage layer.

In recent years, the implantation of tissue engineering stents has been considered as an effective strategy for the treatment of osteochondral defects. There have been many methods for repairing cartilage defects based on biomaterials, and these methods have certain curative effects on osteochondral injuries. However, the research focus is mainly on the cartilage area of the defect site, and the subchondral bone and calcified cartilage area are often ignored. Now it has been recognized that subchondral bone injury can have an important impact on the occurrence and development of degenerative joint diseases. Therefore, in order to complete the repair of osteochondral defects, healthy subchondral bone must be regenerated at the defect site. Nowadays, the development of bioactive multilayer stents for bone and cartilage regeneration is considered an ideal strategy. These materials are usually composed of separate cartilage layer and bone repair layer, and these stents can not really solve the problem of multi-layer repair of osteochondral tissue. In addition, improving the stability of the double-layer stent and achieving a good interface between different layers remains a major challenge.

### SUMMARY OF THE INVENTION

Recombinant collagen is a new type of genetic engineering protein. It can be obtained by reverse transcription of human collagen mRNA into cDNA through recombinant Escherichia coli BL21. After enzyme digestion, specific suture and connection, it is introduced into Escherichia coli for high-density fermentation, and then separated and purified. In addition to the advantages of collagen, recombinant collagen also has the advantages of good water solubility, low immunogenicity, good product stability and no potential virus. Because of these characteristics, recombinant collagen has been widely used in various aspects of biomedical engineering, including the preparation of soft tissue fillers, hemostatic sponges and vascular stents. Sodium hyaluronate is a linear polysaccharide composed of 250-25000 repeated disaccharide units. It is the most abundant component in cartilage and an important component of aggregating proteoglycan, which organizes cartilage cytoplasmic matrix into elastic structure. Therefore, sodium hyaluronate based hydrogels are one of the most promising natural biomaterials for osteochondral tissue engineering and cartilage tissue engineering applications. According to the mineral composition of the calcified cartilage interface, hydroxyapatite is the best choice for stent materials at the osteochondral interface.

In view of the problems existing in the existing cartilage tissue repair stent, the present application provides a double-layer osteochondral tissue repair stent and a preparation method thereof. Specifically, the technical scheme provided by the present application is as follows:

1. A method for preparing a double-layer osteochondral tissue repair stent, which is characterized in that, the method comprises:
preparing a first feed solution comprising recombinant collagen, sodium hyaluronate and hydroxyapatite;
preparing a second feed solution comprising recombinant collagen and sodium hyaluronate; freeze drying the first feed solution and the second feed solution to form a gel-like double-layer structure; and
adding a crosslinking agent to the gel-like double-layer structure for crosslinking.
2. The method according to item 1, which is characterized in that, the concentrations of the recombinant collagen in the first feed solution and the second feed solution are different.
3. The method according to item 2, which is characterized in that, the concentration of the recombinant collagen in the first feed solution is higher than that in the second feed solution.
4. The method according to item 3, which is characterized in that, the concentration range of the recombinant collagen in the first feed solution is 90˜120 mg/mL, and the concentration range of the recombinant collagen in the second feed solution is 60˜90 mg/mL.
5. The method according to item 1, which is characterized in that, the concentration of sodium hyaluronate in the first feed solution is the same as that in the second feed solution.
6. The method according to item 5, which is characterized in that, the concentration range of sodium hyaluronate in the first feed solution and the second feed solution is 8-15 mg/mL.
7. The method according to item 1, which is characterized in that, the concentration range of hydroxyapatite in the first feed solution is 30˜60 mg/mL.
8. The method according to item 1, which is characterized in that, the process of freeze drying the first feed solution and the second feed solution to form a gel-like double-layer structure comprises:
pouring the first feed solution into a mold, cooling and standing to obtain a first gel-like body;
pouring the second feed solution into the upper layer of the first gel-like body and standing to obtain a gel-like double-layer structure.
9. The method according to item 1, which is characterized in that, the process of freeze drying the first feed solution and the second feed solution to form a gel-like double-layer structure comprises:
pouring the second feed solution into a mold, cooling and standing to obtain a second gel-like body;
pouring the first feed solution into the upper layer of the second gel-like body and standing to obtain a gel-like double-layer structure.
10. The method according to any one of items 1 to 9, which is characterized in that, the molecular weight of the recombinant collagen is 80 kD-110 kD.
11. The method according to any one of items 1 to 9, which is characterized in that, the molecular weight of sodium hyaluronate is 80 kD-150 kD.
12. The method according to any one of items 1 to 9, which is characterized in that, the cross-linking agent is 1-(3-dimethylaminopropyl)-3-ethylcarbodiimide hydrochloride.
13. The method according to any one of items 1 to 9, which is characterized in that, the process of adding a crosslinking agent to the gel-like double-layer structure for crosslinking comprises: immersing the gel-like double-layer structure in a 1-(3-dimethylaminopropyl)-3-ethylcarbodiimide hydrochloride solution.
14. The method according to any one of items 1 to 9, which is characterized in that, the concentration of the 1-(3-dimethylaminopropyl)-3-ethylcarbodiimide hydrochloride solution is 30-60 mmol/mL.
15. A double-layer osteochondral tissue repair stent prepared by the method according to any one of items 1 to 14.
16. A double-layer osteochondral tissue repair stent, which is characterized in that, the double-layer osteochondral tissue repair stent comprises:
a first layer, the first layer is made of raw materials comprising recombinant collagen, sodium hyaluronate and hydroxyapatite; and
a second layer, the second layer is made of raw materials comprising recombinant collagen and sodium hyaluronate.
17. The double-layer osteochondral tissue repair stent according to item 16, which is characterized in that, the porosity of the first layer is 80%-97%, and the porosity of the second layer is 58%-86%.
18. The double-layer osteochondral tissue repair stent according to item 16, which is characterized in that, and the aperture of the first layer is 50-80 μm, the aperture of the second layer is 100-200 μm.
19. The double-layer osteochondral tissue repair stent according to item 16, which is characterized in that, the thickness of the first layer is 2-4 mm, and the thickness of the second layer is 3-6 mm.

The double-layer osteochondral tissue repair stent prepared by the present application has excellent mechanical properties, good biocompatibility and suitable degradation rate, and the stent material can be reused as raw material for new bone generation after degradation, so as to achieve the repair of osteochondral tissue.

### BRIEF DESCRIPTION OF THE DRAWINGS

FIG. 1 is the structural diagram of the double-layer osteochondral tissue repair stent of the present application.

The appended drawing reference signs: 1—the first layer, 2—the second layer.

### DETAIL DESCRIPTION OF THE INVENTION

The present application relates to a method for preparing a double-layer osteochondral tissue repair stent comprising:

preparing a first feed solution, the first feed solution comprises recombinant collagen, sodium hyaluronate and hydroxyapatite;
preparing a second feed solution, the second feed solution comprises recombinant collagen and sodium hyaluronate;
freeze drying the first feed solution and the second feed solution to form a gel-like double-layer structure; and
adding a crosslinking agent to the gel-like double-layer structure for crosslinking.

Wherein, recombinant collagen is a new type of genetic engineering protein. It can be obtained by reverse transcription of human collagen mRNA into cDNA through recombinant Escherichia coli BL21. After enzyme digestion, specific suture and connection, it is introduced into Escherichia coli for high-density fermentation, and then separated and purified. In this specification, recombinant collagen refers to the recombinant collagen described in claim 1 of the Chinese patent application publication CN1371919A, which has a triple chain and triple helix structure, and can be prepared using, for example, the genetic engineering expression method disclosed in the Chinese patent application publication CN1371919A.

In a specific embodiment, the concentrations of the recombinant collagen in the first feed solution and the second feed solution are different. Furthermore, the concentration of the recombinant collagen in the first feed solution is higher than that in the second feed solution.

In a specific embodiment, the concentration range of the recombinant collagen in the first feed solution is 90˜120 mg/mL, for example, it can be 90 mg/mL, 95 mg/mL, 100 mg/mL, 105 mg/mL, 110 mg/mL, 115 mg/mL, or 120 mg/mL. The concentration range of the recombinant collagen in the second feed solution is 60˜90 mg/mL, for example, it can be 60 mg/mL, 65 mg/mL, 70 mg/mL, 75 mg/mL, 80 mg/mL, 85 mg/mL, or 90 mg/mL.

In a specific embodiment, the concentration of sodium hyaluronate in the first feed solution is the same as that in the second feed solution. Furthermore, the concentration range of sodium hyaluronate in the first feed solution and the second feed solution is 8-15 mg/mL, for example, it can be 8 mg/mL, 9 mg/mL, 10 mg/mL, 11 mg/mL, 12 mg/mL, 13 mg/mL, 14 mg/mL, or 15 mg/mL.

In a specific embodiment, the concentration range of hydroxyapatite in the first feed solution is 30-60 mg/mL, for example, it can be 30 mg/mL, 35 mg/mL, 40 mg/mL, 45 mg/mL, 50 mg/mL, 55 mg/mL, or 60 mg/mL.

In a specific embodiment, the process of freeze drying the first feed solution and the second feed solution to form a gel-like double-layer structure comprises: pouring the first feed solution into a mold, cooling and standing to obtain a first gel-like body; and pouring the second feed solution into the upper layer of the first gel-like body and standing to obtain a gel-like double-layer structure. That is, the first feed solution forms the bottom layer of the gel-like double-layer structure, that is, the gel-like body formed first.

In a specific embodiment, the process of freeze drying the first feed solution and the second feed solution to form a gel-like double-layer structure comprises: pouring the second feed solution into a mold, cooling and standing to obtain a second gel-like body; and pouring the first feed solution into the upper layer of the second gel-like body and standing to obtain a gel-like double-layer structure. That is, the second feed solution forms the bottom layer of the gel-like double-layer structure, that is, the gel-like body formed first.

In a specific embodiment, the molecular weight of the recombinant collagen is 80 kD˜110 kD, for example, it can be 80 kD, 85 kD, 90 kD, 95 kD, 97 kD, 100 kD, or 110 kD.

In a specific embodiment, the molecular weight of the sodium hyaluronate is 80 kD˜150 kD, for example, it can be 80 kD, 90 kD, 100 kD, 110 kD, 120 kD, 130 kD, 140 kD, or 150 kD.

In a specific embodiment, the cross-linking agent is 1-(3-dimethylaminopropyl)-3-ethylcarbodiimide hydrochloride. Furthermore, the concentration of the 1-(3-dimethylaminopropyl)-3-ethylcarbodiimide hydrochloride solution is 30-60 mmol/mL. For example, it can be 30 mmol/mL, 40 mmol/mL, 50 mmol/mL, or and 60 mmol/mL. The cross-linking condition is cross-linking for 24-72 h, and cleaning with pure water for 3-5 times.

In a specific embodiment, the method further comprises freeze drying, packaging and sterilization of the double-layer gel after crosslinking the double-layer gel.

The present application also provides a double-layer cartilage tissue repair stent prepared by the above method.

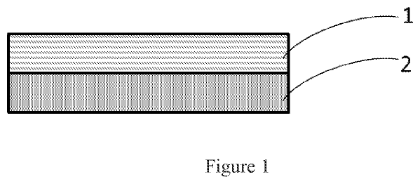

The present application also provides a double-layer osteochondral tissue repair stent, as shown in FIG. 1, the double-layer osteochondral tissue repair stent comprises a first layer 1 and a second layer 2, wherein the first layer 1 is made of raw materials comprising recombinant collagen, sodium hyaluronate and hydroxyapatite, and the second layer 2 is made of raw materials comprising recombinant collagen and sodium hyaluronate.

In a specific embodiment, the porosity of the first layer 1 is 80%˜97%.

In a specific embodiment, the porosity of the second layer 2 is 58%˜86%.

In a specific embodiment, the aperture of the first layer 1 is 100-200 μm. For example, it can be 100 μm, 110 μm, 120 μm, 130 μm, 140 μm, 150 μm, 160 v, 170 v, 180 μm, 190 μm or 200 μm.

In a specific embodiment, the aperture of the second layer 2 is 50-80 μm. For example, it can be 50 μm, 60 μm, 70 μm or 80 μm.

The thickness and width of the first layer 1 and the second layer 2 can be adjusted according to actual needs. In a specific embodiment, the thickness of the first layer 1 is 2-4 mm, and the thickness of the second layer 2 is 3-6 mm.

### EXAMPLE

### Example 1

The double-layer osteochondral tissue repair stent in this example simulates the physiological structure and composition of natural osteochondral, and the upper layer uses recombinant collagen and sodium hyaluronate to repair the cartilage layer; the lower layer uses recombinant collagen and sodium hyaluronate as the organic phase and nano hydroxyapatite as the inorganic phase to repair the subchondral bone layer. After freeze-drying, it is prepared by crosslinking under the action of a crosslinking agent, which is a 95% ethanol solution of 1-(3-dimethylaminopropyl)-3-ethylcarbodiimide hydrochloride.

The method for preparing the double-layer osteochondral tissue repair stent of the example specifically included:

(1) the first feed solution was prepared, wherein the first feed solution comprised recombinant collagen, sodium hyaluronate and hydroxyapatite. Specifically, the recombinant collagen, sodium hyaluronate and hydroxyapatite were added to the non heat source water, and stirred evenly to obtain the first feed solution was obtained. The concentration of the recombinant collagen was 100 mg/mL, the molecular weight of the recombinant collagen was 97 kD, the concentration of sodium hyaluronate was 10 mg/mL, the molecular weight of sodium hyaluronate was 100 kD, and the concentration of hydroxyapatite was 50 mg/mL.
(2) the second feed solution was prepared, wherein the second feed solution comprised recombinant collagen and sodium hyaluronate. Specifically, the recombinant collagen and sodium hyaluronate were added to the non heat source water, and stirred evenly to obtain the second feed solution. The concentration of the recombinant collagen was 80 mg/mL, the molecular weight of the recombinant collagen was 97 kD, the concentration of sodium hyaluronate was 10 mg/mL, and the molecular weight of sodium hyaluronate was 100 kD.
(3) 1 mL of the first feed solution was poured into a Φ15*10 columnar mold, and cooled at 4° C. for 30 mins to obtain a first gel; 2 ml of the second feed solution was poured into the upper layer of the first gel-like body, and stood at room temperature for 40 min to obtain a gel-like double-layer structure.
(4) the obtained gel-like bilayer structure was freeze-dried, and then crosslinked with ethanol (95%) solution of 1-(3-dimethylaminopropyl)-3-ethylcarbodiimide hydrochloride for 36 hours (30˜60 mmol/ml), wherein the amount of crosslinking agent was 50 mmol/ml. After the cross-linking reaction, it was cleaned for 3-5 times with purified water to remove the residual cross-linking agent. After cleaning, the stent was freeze-dried, packaged and sterilized to obtain a double-layer osteochondral tissue repair stent.

### Example 2

The difference between Example 2 and Example 1 was that in the first feed solution, the concentration of the recombinant collagen was 95 mg/mL, the molecular weight of the recombinant collagen was 100 kD, the concentration of sodium hyaluronate was 9 mg/mL, the molecular weight of sodium hyaluronate was 90 kD, and the concentration of hydroxyapatite was 45 mg/mL; in the second feed solution, the concentration of the recombinant collagen was 85 mg/mL, the molecular weight of the recombinant collagen was 100 kD, the concentration of sodium hyaluronate was 9 mg/mL, and the molecular weight of sodium hyaluronate was 90 kD. Other reaction conditions were the same as those in Example 1. See Table 1 for details.

### Example 3

The difference between Example 3 and Example 1 was that in the first feed solution, the concentration of the recombinant collagen was 105 mg/mL, the molecular weight of the recombinant collagen was 95 kD, the concentration of sodium hyaluronate was 12 mg/mL, the molecular weight of sodium hyaluronate was 110 kD, and the concentration of hydroxyapatite was 55 mg/mL; in the second feed solution, the concentration of the recombinant collagen was 75 mg/mL, the molecular weight of the recombinant collagen was 95 kD, the concentration of sodium hyaluronate was 12 mg/mL, and the molecular weight of sodium hyaluronate was 110 kD. Other reaction conditions were the same as those in Example 1. See Table 1 for details.

### Example 4

The difference between Example 4 and Example 1 was that in the first feed solution, the concentration of the recombinant collagen was 120 mg/mL, the molecular weight of the recombinant collagen was 105 kD, the concentration of sodium hyaluronate was 8 mg/mL, the molecular weight of sodium hyaluronate was 80 kD, and the concentration of hydroxyapatite was 40 mg/mL; in the second feed solution, the concentration of the recombinant collagen was 70 mg/mL, the molecular weight of the recombinant collagen was 105 kD, the concentration of sodium hyaluronate was 8 mg/mL, and the molecular weight of sodium hyaluronate was 80 kD. Other reaction conditions were the same as those in Example 1. See Table 1 for details.

### Example 5

The difference between Example 5 and Example 1 was that in the first feed solution, the concentration of the recombinant collagen was 115 mg/mL, the molecular weight of the recombinant collagen was 80 kD, the concentration of sodium hyaluronate was 15 mg/mL, the molecular weight of sodium hyaluronate was 120 kD, and the concentration of hydroxyapatite was 30 mg/mL; In the second feed solution, the concentration of the recombinant collagen was 90 mg/mL, the molecular weight of the recombinant collagen was 80 kD, the concentration of sodium hyaluronate was 15 mg/mL, and the molecular weight of sodium hyaluronate was 120 kD. Other reaction conditions were the same as those in Example 1. See Table 1 for details.

### Example 6

The difference between Example 6 and Example 1 was that in the first feed solution, the concentration of the recombinant collagen was 110 mg/mL, the molecular weight of the recombinant collagen was 110 kD, the concentration of sodium hyaluronate was 14 mg/mL, the molecular weight of sodium hyaluronate was 130 kD, and the concentration of hydroxyapatite was 60 mg/mL; in the second feed solution, the concentration of the recombinant collagen was 65 mg/mL, the molecular weight of the recombinant collagen was 110 kD, the concentration of sodium hyaluronate was 14 mg/mL, and the molecular weight of sodium hyaluronate was 130 kD. Other reaction conditions were the same as those in Example 1. See Table 1 for details.

### Example 7

The difference between Example 7 and Example 1 was that in the first feed solution, the concentration of the recombinant collagen was 90 mg/mL, the molecular weight of the recombinant collagen was 85 kD, the concentration of sodium hyaluronate was 13 mg/mL, the molecular weight of sodium hyaluronate was 150 kD, and the concentration of hydroxyapatite was 35 mg/mL; in the second feed solution, the concentration of the recombinant collagen was 60 mg/mL, the molecular weight of the recombinant collagen was 85 kD, the concentration of sodium hyaluronate was 13 mg/mL, and the molecular weight of sodium hyaluronate was 150 kD. Other reaction conditions were the same as those in Example 1. See Table 1 for details.

### Comparative Example 1

The difference between Comparative Example 1 and Example 1 was that in the first feed solution, the concentration of the recombinant collagen was 80 mg/mL, the molecular weight of the recombinant collagen was 120 kD, the concentration of sodium hyaluronate was 5 mg/mL, the molecular weight of sodium hyaluronate was 130 kD, and the concentration of hydroxyapatite was 70 mg/mL; in the second feed solution, the concentration of the recombinant collagen was 100 mg/mL, the molecular weight of the recombinant collagen was 120 kD, the concentration of sodium hyaluronate was 5 mg/mL, and the molecular weight of sodium hyaluronate was 130 kD. Other reaction conditions were the same as those in Example 1. See Table 1 for details.

### Comparative Example 2

The difference between Comparative Example 2 and Example 1 was that in the first feed solution, the concentration of the recombinant collagen was 130 mg/mL, the molecular weight of the recombinant collagen was 70 kD, the concentration of sodium hyaluronate was 18 mg/mL, the molecular weight of sodium hyaluronate was 170 kD, and the concentration of hydroxyapatite was 40 mg/mL; in the second feed solution, the concentration of the recombinant collagen was 60 mg/mL, the molecular weight of the recombinant collagen was 70 kD, the concentration of sodium hyaluronate was 18 mg/mL, and the molecular weight of sodium hyaluronate was 170 kD. Other reaction conditions were the same as those in Example 1. See Table 1 for details.

### Comparative Example 3

The difference between Comparative Example 3 and Example 1 was that in the first feed solution, the concentration of the recombinant collagen was 90 mg/mL, the molecular weight of the recombinant collagen was 90 kD, the concentration of sodium hyaluronate was 10 mg/mL, the molecular weight of sodium hyaluronate was 60 kD, and the concentration of hydroxyapatite was 20 mg/mL; in the second feed solution, the concentration of the recombinant collagen was 110 mg/mL, the molecular weight of the recombinant collagen was 90 kD, the concentration of sodium hyaluronate was 10 mg/mL, and the molecular weight of sodium hyaluronate was 60 kD. Other reaction conditions were the same as those in Example 1. See Table 1 for details.

TABLE 1
Specific reaction conditions of Examples and Comparative Examples

| | | | | | Molecular | |
|---|---|---|---|---|---|---|
| | Concentration of | Concentration of | Concentration | | weight of | Molecular |
| | the recombinant | the recombinant | of sodium | Concentration of | the | weight of |
| | collagen in the | collagen in the | hyaluronate in | hydroxyapatite in | recombinant | sodium |
| | first feed solution | second feed | feed solution | the first feed | collagen | hyaluronate |
| | (mg/mL) | solution (mg/mL) | (mg/mL) | solution (mg/mL) | (kD) | (kD) |
| Example 1 | 100 | 80 | 10 | 50 | 97 | 100 |
| Example 2 | 95 | 85 | 9 | 45 | 100 | 90 |
| Example 3 | 105 | 75 | 12 | 55 | 95 | 110 |
| Example 4 | 120 | 70 | 8 | 40 | 105 | 80 |
| Example 5 | 115 | 90 | 15 | 30 | 80 | 120 |
| Example 6 | 110 | 65 | 14 | 60 | 110 | 130 |
| Example 7 | 90 | 60 | 13 | 35 | 85 | 150 |
| Comparative | 80 | 100 | 5 | 70 | 120 | 130 |
| example 1 | | | | | | |
| Comparative | 130 | 60 | 18 | 40 | 70 | 170 |
| example 2 | | | | | | |
| Comparative | 90 | 110 | 10 | 20 | 90 | 60 |
| example 3 | | | | | | |

Performances of the double-layer osteochondral tissue repair stent samples prepared from Examples 1-7 and the Comparative Examples 1-3 were tested.

### (1) Mechanical Property Test

In order to verify the mechanical properties of the prepared double-layer osteochondral tissue repair stent material, the double-layer osteochondral tissue repair stent samples prepared in the above Examples and Comparative Examples were selected and tested by the electronic universal material testing machine (INSTRON 5565) using a 500 N sensor. Specifically, the double-layer osteochondral stent sample was made into a cylinder with a diameter of 15 mm and a height of 10 mm. The compression performance of the tissue engineered cartilage stent was measured at a loading rate of 10 mm/min. After the measurement, the compression stress, compression strain, compression elastic modulus and other data were collected. The elastic modulus (E) is determined by the slope of the stress-strain curve obtained for each sample.

### (2) Cytotoxicity Test

In order to verify the biological compatibility of the prepared double-layer osteochondral repair stent material, the double-layer osteochondral tissue repair stent material samples prepared in the above Examples and Comparative Examples were selected for relevant cytotoxicity tests.

The cytotoxicity test of stent materials was to evaluate the potential hazards of materials. This test used MTT method to detect the cytotoxicity of stent materials. First, the extract solution of the stent material was prepared, that is, a 100 mg/mL stent extract solution was prepared by selecting 2.0 g of sterile double-layer osteochondral repair stent material sterilized by Co-60 radiation prepared in the above examples of the present application, adding 20 mL DMEM complete culture solution, and extracting in a incubator at 37° C. for 72±2 hours.

HBMSC cells were cultured at 37° C. with 5.0% CO₂ concentration. When the second generation hBMSC cells grew to 70%, they were digested with 3 mL trypsin solution, and inoculated into 96 well cell culture plates with a cell density of 3×10⁴ cells/mL, and inoculated 100 μL cells per well. The cell culture plate with cells was put into the incubator for 24 hours, the culture medium was sucked out, and every 8 holes were taken as a group of parallel samples. The control group used 100 μL complete culture medium for cell culture, and the test group used 100 μL of the extract solution of stent material for culture cells. After 7 days of culture, the culture medium was sucked out, and 50 μL MTT solution and 100 μL fresh complete culture medium were added to each well. The plate was incubated in CO₂ incubator at 37° C. for 3 hours, the culture medium was took out and 150 μL DMSO was added, incubated in a shaking table for 15-20 minutes, and the absorbance value was measured at 450 nm with a microplate reader to calculate the relative proliferation rate of hBMSC cells.

### (3) Osteochondral Defect Model Repair Test

In order to verify the repair effect of the double-layer osteochondral stent prepared by the present application on the osteochondral defects, the double-layer osteochondral stent prepared by the above Examples and Comparative Examples were selected for animal experiments, specifically:

Under general anesthesia, New Zealand white rabbits were used to grind osteochondral defects (3.0 mm in diameter and 5.0 mm in depth) in the trochlear groove of their right legs with a dental grinder, and then porous double-layer osteochondral stents with the same size as the defects were implanted into the defects. The rabbits were randomly divided into two groups: the stent group and the control group (only defects). The rabbits were euthanized 12 weeks after the operation, the tissue repair was observed, and the percentage of osteochondral repair was calculated.

The results of the above mechanical property test, cytotoxicity test and osteochondral defect model repair test are shown in Table 2.

TABLE 2
Different test results for Examples and Comparative Examples

| | | Relative cell | Repair rate |
|---|---|---|---|
| | Elastic | proliferation | of animal |
| | modulus(MPa) | rate(%) | osteochondrosis (%) |
| Example 1 | 2.04 | 120 | 96 |
| Example 2 | 1.98 | 115 | 92 |
| Example 3 | 1.96 | 120 | 92 |
| Example 4 | 1.94 | 110 | 90 |
| Example 5 | 1.82 | 108 | 90 |
| Example 6 | 2.15 | 95 | 89 |
| Example 7 | 2.08 | 105 | 87 |
| Comparative | 2.30 | 91 | 80 |
| example 1 | | | |
| Comparative | 2.25 | 93 | 78 |
| example 2 | | | |
| Comparative | 1.76 | 90 | 82 |
| example 3 | | | |

Through the analysis of the results of mechanical property test, cytotoxicity test and osteochondral defect repair test, it can be seen from Table 2 that the double-layer osteochondral tissue repair stent of Examples 1-7 can be compressed to more than 70%, and its elastic modulus is about 2 MPa. After 20 cycles of compression, the double-layer osteochondral stent material has good recovery performance, and its mechanical properties fully meet the human needs.

Through cytological experiments, the results show that the relative cell proliferation rate of the extract of the double-layer osteochondral tissue repair stent material prepared in Examples 1-7 of the present application reaches more than 100% or nearly 100%, and the cytotoxicity evaluation is grade 0.

The animal experiment of the double-layer osteochondral tissue repair stent prepared by the present application for repairing osteochondral defects shows that: the repair rates of animal osteochondrosis of the sterile porous bone repair stent prepared in Examples 1-7 are significantly higher than that of the stent prepared in Comparative Examples, especially the repair rates of animal osteochondrosis in Examples 1-5 are more than 90%, which show that the animal osteochondral defects are filled with uniform cartilage like tissue, the new tissue is well connected with the surrounding normal cartilage, indicating that these double-layer osteochondral tissue repair stents have a good repair effect on osteochondral defects.

The present application accepts various modified and replaceable forms. The specific embodiments have been shown in the drawings with the help of Examples and have been described in detail in the present application. However, the present application is not intended to be limited to the specific form of disclosure. On the contrary, the application is intended to include all modified forms, equivalents, and substitutions within the scope of the application. The scope of the application is defined by the appended claims and their legal equivalents.

The numerical range listed in the present application includes the data of two endpoints of the numerical range, as well as each specific value in the numerical range, and the value can be arbitrarily combined with the endpoint to form a new small range.

1. A method for preparing a double-layer osteochondral tissue repair stent, which is characterized in that, the method comprises:
preparing a first feed solution comprising recombinant collagen, sodium hyaluronate and hydroxyapatite;
preparing a second feed solution comprising recombinant collagen and sodium hyaluronate;
freeze drying the first feed solution and the second feed solution to form a gel-like double-layer structure; and
adding a crosslinking agent to the gel-like double-layer structure for crosslinking.

2. The method according to claim 1, which is characterized in that, the concentrations of the recombinant collagen in the first feed solution and the second feed solution are different.

3. The method according to claim 2, which is characterized in that, the concentration of the recombinant collagen in the first feed solution is higher than that in the second feed solution.

4. The method according to claim 3, which is characterized in that, the concentration range of the recombinant collagen in the first feed solution is 90˜120 mg/mL, and the concentration range of the recombinant collagen in the second feed solution is 60˜90 mg/mL.

5. The method according to claim 1, which is characterized in that, the concentration of sodium hyaluronate in the first feed solution is the same as that in the second feed solution.

6. The method according to claim 5, which is characterized in that, the concentration range of sodium hyaluronate in the first feed solution and the second feed solution is 8-15 mg/mL.

7. The method according to claim 1, which is characterized in that, the concentration range of hydroxyapatite in the first feed solution is 30˜60 mg/mL.

8. The method according to claim 1, which is characterized in that, the process of freeze drying the first feed solution and the second feed solution to form a gel-like double-layer structure comprises:
pouring the first feed solution into a mold, cooling and standing to obtain a first gel-like body; and
pouring the second feed solution into the upper layer of the first gel-like body and standing to obtain a gel-like double-layer structure.

9. The method according to claim 1, which is characterized in that, the process of freeze drying the first feed solution and the second feed solution to form a gel-like double-layer structure comprises:
pouring the second feed solution into a mold, cooling and standing to obtain a second gel-like body; and
pouring the first feed solution into the upper layer of the second gel-like body and standing to obtain a gel-like double-layer structure.

10. The method according to claim 1, which is characterized in that, the molecular weight of the recombinant collagen is 80 kD-110 kD.

11. The method according to claim 1, which is characterized in that, the molecular weight of sodium hyaluronate is 80 kD-150 kD.

12. The method according to claim 1, which is characterized in that, the cross-linking agent is 1-(3-dimethylaminopropyl)-3-ethylcarbodiimide hydrochloride.

13. The method according to claim 1, which is characterized in that, the process of adding a crosslinking agent to the gel-like double-layer structure for crosslinking comprises:
immersing the gel-like double-layer structure in a 1-(3-dimethylaminopropyl) ethylcarbodiimide hydrochloride solution.

14. The method according to claim 13, which is characterized in that, the concentration of the 1-(3-dimethylaminopropyl)-3-ethylcarbodiimide hydrochloride solution is 30-60 mmol/mL.

15. A double-layer osteochondral tissue repair stent prepared by the method according to claim 1.

16. A double-layer osteochondral tissue repair stent, which is characterized in that, the double-layer osteochondral tissue repair stent comprises:
a first layer, the first layer is made of raw materials comprising recombinant collagen, sodium hyaluronate and hydroxyapatite; and
a second layer, the second layer is made of raw materials comprising recombinant collagen and sodium hyaluronate.

17. The double-layer osteochondral tissue repair stent according to claim 16, which is characterized in that, the porosity of the first layer is 80%-97%, and the porosity of the second layer is 58%-86%.

18. The double-layer osteochondral tissue repair stent according to claim 16, which is characterized in that, the aperture of the first layer is 50-80 μm, and the aperture of the second layer is 100-200 μm.

19. The double-layer osteochondral tissue repair stent according to claim 16, which is characterized in that, the thickness of the first layer is 2-4 mm, and the thickness of the second layer is 3-6 mm.
